(19) United States
(12) Patent Application Publication
(10) Pub. No.: US 2019/0298655 A1
(43) Pub. Date: Oct. 3, 2019

(54) ANTI-ALCOHOL-INDUCED DOSE DUMPING TABLET BASED ON POLYVINYL ALCOHOL

(71) Applicant: MERCK PATENT GMBH, DARMSTADT (DE)

(72) Inventors: Mengyao ZHENG, Darmstadt (DE); Alessandro ELIA, Darmstadt (DE)

(73) Assignee: MERCK PATENT GMBH, Darmstadt (DE)

(21) Appl. No.: 16/348,010

(22) PCT Filed: Nov. 6, 2017
(86) PCT No.: PCT/EP2017/078268

§ 371 Date: May 7, 2019

(30) Foreign Application Priority Data
Nov. 7, 2016 (EP) 16197613.9

(51) Int. Cl. A61K 9/20

(52) U.S. Cl. CPC A61K 9/2027; A61K 9/2095

(57) ABSTRACT

The present invention relates to a directly shaped tablet from extrudate based on polyvinyl alcohol (PVA), which can be used for pharmaceutical products, and that due to its improved properties can be better easily shaped into tablets, which show anti-alcohol-induced dose dumping effect. Furthermore, this invention refers to pharmaceutical tablets composition comprising extruded polyvinyl alcohol as carrier matrix and can improve the solubility of API.

The present invention relates to a downstream formulation process of hot melt extrusion including the process steps from producing the extrudate to directly shaped tablets based on polyvinyl alcohol (PVA) as excipient with anti-alcohol-induced dose dumping effect. Furthermore, this invention refers also to the compositions of directly shaped tablets which are suitable to deliver pharmaceutical active ingredients in a sustained release kinetic from a composition comprising polyvinyl alcohol as carrier matrix.

Accordingly, the invention includes both this composition with PVA as a carrier matrix and its use.

### TECHNICAL FIELD

Solid dispersions are defined as being a dispersion of one or more active ingredients in an inert solid matrix and can broadly classified as those containing a drug substance in the crystalline state or in the amorphous state [Chiou W. L., Riegelman S. Pharmaceutical applications of Solid dispersion systems; J. Pharm Sci. 1971, 60 (9), 1281-1301]. In order to achieve a more consistent dosage rate of the active ingredient in pharmaceutical formulations, it is useful when the active ingredient is present as a homogeneous solid dispersion or as solution in a carrier. Solid dispersions containing pharmaceutical active ingredients in the crystalline state provide dissolution enhancement by simply decreasing surface tension, reducing agglomeration, and improving wettability of the active substance [Sinswat P., et al.; Stabilizer choice for rapid dissolving high potency itraconazole particles formed by evaporative precipitation into aqueous solution; Int. J. of Pharmaceutics, (2005) 302; 113-124]. While crystalline systems are more thermodynamically stable than their amorphous counterparts, the crystalline structure must be interrupted during the dissolution process, requiring energy. Solid dispersions containing an active ingredient, this means a drug, dissolved at the molecular level, known as amorphous solid solutions, can result in a significant increase in dissolution rate and extent of supersaturation [DiNunzio J. C. et al. III Amorphous compositions using concentration enhancing polymers for improved bioavailability of itraconazole; Molecular Pharmaceutics (2008); 5(6):968-980].

While these systems have several advantages, physical instability can be problematic due to molecular mobility and the tendency of the drug to recrystallize. Polymeric carriers with high glass transition temperatures seem to be well suited to stabilize these systems by limiting molecular mobility.

As such, solid dispersions can be created by a number of methods, including, but not limited to, spray-drying, melt extrusion, and thermos-kinetic compounding.

Although hot melt extrusion (HME), a fusion processing technique, has been used in the food and plastics industry for more than a century, it has only recently gained acceptance in the pharmaceutical industry for the preparation of formulations comprising active ingredients processed by extrusion. And now, HME has been introduced as pharmaceutical manufacturing technology and has become a well-known process with benefits like continuous and effective processing, limited number of process steps, solvent free process etc.

During hot melt extrusion the active ingredients are mixed with and embedded in excipients, such as polymers and plasticizers. Furthermore, drug substances are exposed to elevated temperatures for a period of time. Although a variety of factors can affect the residence time distribution of an extruded substance, these times typically fall within the 1- to 2-min range (Breitenbach J., Melt extrusion: from process to drug delivery technology. Eur J Pharm Biopharm. (2002), 54, 107-117).

Therefore, as carriers for the application of (hot) melt extrusion, the polymers should have suitable properties such us thermoplasticity, suitable glass transition temperature or melting point, thermostability at required processing temperature, no unexpected chemical interaction with active ingredients etc. In this context, polyvinyl alcohol (PVA) is an excellent compound, which is suitable for (hot) melt extrusion, as carrier for pharmaceutically active ingredients. Polyvinyl alcohol (PVA) is a synthetic water-soluble polymer that possesses excellent film-forming, adhesive, and emulsifying properties. It is prepared from polyvinyl acetate, where the functional acetate groups are either partially or completely hydrolyzed to alcohol functional groups. As the degree of hydrolysis increases, the solubility of the polymer in aqueous media increases, but also the crystallinity of the polymer increases. In addition to this, the glass transition temperature varies depending on its degree of hydrolysis.

During hot melt extrusion, mixtures of active ingredients, thermoplastic excipients, and other functional processing aids, are heated and softened or melted inside of an extruder and extruded through nozzles into different forms. The obtained extrudate can be cut down into small beads or milled into fine powder, or directly shaped into tablets.

In hot melt extrusion, thermoplastic polymer PVA may be mixed with a pharmaceutical active substance (API) and optional inert excipients and further additives, such as plasticizer. The mixture is fed into rotating screws that convey the powder into a heated zone where shear forces are imparted into the mixture, compounding the materials until a molten mass is achieved. The extrudate with solid dispersed API can be directly shaped into tablets. The solubility of API can be improved in the final dosage form of the directly shaped tablet. In different combinations of the compositions, the PVA-based direct-molded tablets may have different release kinetics.

U.S. Pat. No. 5,456,923 A provides a process for producing a solid dispersion, which overcomes disadvantages of the conventional production technology for solid dispersions. The process comprises employing a twin-screw extruder in the production of a solid dispersion. In accordance with this, a solid dispersion can be expediently produced without heating a drug and a polymer up to or beyond their melting points and without using an organic solvent for dissolving both components and the resulting solid dispersion has excellent performance characteristics. The process claims a polymer that is natural or synthetic and can be employed as a raw material where the polymer's functions are not adversely affected by passage through the twin screw extruder.

EP 2 105 130 A1 describes a pharmaceutical formulation comprising a solid dispersion having an active substance embedded in a polymer in amorphous form, and an external polymer as a recrystallization inhibitor independently of the solid dispersion. The external polymer is claimed as a solution stabilizer. The active substance should be sparingly soluble or less sparingly soluble in water. Thermoplastic polymers are claimed as drug carriers to form a solid dispersion. It is claimed that the solid dispersion is obtained by melt extrusion. The process comprises melting and mixing the polymer and the active ingredient, cooling, grinding, mixing with the external polymer, and producing a pharmaceutical formulation. It is claimed that the melting is carried out at a temperature below the melting point of the drug. It is also claimed that the melting is carried out at a temperature above the T_{g} or melting point of the polymer, but from 0.1-5° C. below the melting point of the API. The melting point of pharmaceutical grades of PVA is normally above 178° C., although the glass transition temperature is in the range of 40-45° C.

### Problem to be Solved

Controlled release formulations, which are intended for once daily administration are designed with a higher unit dose of the drug than conventional formulations. Therefore, it is essential that retardation properties have to be tightly controlled to ensure that a rapid release of the drug, or dose-dumping, cannot occur especially with simultaneous alcohol consumption. It is known that alcohol has an influence on the excretion of drugs from drug formulations, like capsules or tablets, which can potentially lead to unintended side effects or toxicity.

Thus, it is an object of the present invention to provide a sustained release drug formulation by which an alcohol induced dose dumping effect from highly dosed, long-acting oral dosage forms is avoided.

This alcohol-induced dose dumping problem is especially critical for active pharmaceutical ingredients (API) from BCS class II and IV, which are poorly water-soluble, but easy alcohol-soluble.

Another object of the present invention is to reduce high contents of binder material in the compressed, drug containing tablet.

Although the milled PVA particles are fine enough, binder materials are still needed, in general in a ratio of 50% by weight of the drug containing composition, if it is compressed into tablets. The high ratio of binder materials limits the contained percentage of solid dispersion based on PVA with the effect that the drug loading efficiency is therefore also limited, because PVA is the functional polymer to formulate a crystalline API into an amorphous state.

Thus, a further problem to be solved by the present invention is to provide a tablet comprising PVA as carrier and having an adapted disintegration time and release of the contained active ingredient (API), which is suitable for sustained release formulations.

Polyvinyl alcohol (PVA) is well known as very hydrophilic polymer and forms in an aqueous medium a gel layer on the surface of the drug containing compressed tablet, which is prepared from a powder composition based on PVA. This gel layer blocks the disintegration of tablet. The normal compressed tablet contains extruded API and PVA and is even more difficult to be disintegrated than the tablet without any API.

Another problem to be solved by the present invention is that poorly water-soluble active pharmaceutical ingredients (API) of classes BCS II and IV tend to recrystallize again in the gel layer. Thus, the gel layer on the surface of tablets, which are compressed from PVA powder, blocks the release of the containing API, and may promote re-crystallization of the API within the tablets, because the API suffers a super saturated state inside of the compressed tablet.

### SUMMARY OF THE INVENTION

The directly shaped tablet of the present invention belongs to one of the final dosage forms of hot melt extrusion technology. Using special equipment, the extrudate with PVA and API can be directly shaped into a tablet form, without milling or compression.

Surprisingly, by experiments an anti-alcohol induced-dose dumping effect is found for the tablets directly molded from PVA extrudate.

Accordingly, the advantages of the tablet prepared according to the invention are first, that this directly molded tablet is anti-dose dumping and secondly, compared to compressed tablets, said tablets are very inexpensive to manufacture, because they can be produced after hot melt extrusion of all ingredients and by subsequent shaping. Moreover, these tablets are long-term stable, what gives the users the benefit to use it for a longer time.

The applied particular polyvinyl alcohol grades fulfilling said conditions are preferably selected preferably from the group PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88.

Accordingly, a PVA grade is subject matter of the present invention, which is suitable as thermoplastic polymer for HME and also suitable for one of the downstream formulation process of HME to prepare directly shaped tablets. In one embodiment of the invention a polyvinyl alcohol as characterized above is extruded and mixed homogeneously with at least one active pharmaceutical ingredient and then directly shaped into tablet form, whereby the received directly shaped tablet is storage and transport-stable. This directly shaped tablet composition may comprise at least one additive selected from the group plasticizer, antioxidants, stabilizing agents, solubility-enhancing agents, and pH control agents.

Thus, the present invention also consists in a method for producing a directly shaped tablet according to the invention, which shows improved properties in view of an anti-alcohol induced dose dumping effect.

In summary, the particular advantage of the present invention is that the obtained directly shaped tablet shows an anti-alcohol induced dose dumping effect and that it can be produced under reduced costs for the material and the formulation processing.

Moreover, compared with traditional compressed tablets, the directly shaped tablets have no disintegration problem and the dissolution kinetic can also be optimized with additional excipients, which can be mixed and extruded together with PVA.

The process according to the present invention includes the steps of
a) physical blending or granulating of the PVA with at least one pharmaceutical active ingredient and optionally with at least one additive selected from the group plasticizer, antioxidants, stabilizing agents, solubility-enhancing agents, and pH control agents, into a homogeneous mixture,
b) hot melt extrusion or melt extrusion and
c) directly shaping the extrudate into a tablet.

This process can be performed particularly well, if polyvinyl alcohol (PVA) is selected from the group PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88.

Thus, a directly shaped tablet from PVA extrudate, which is characterized as disclosed herein and which is obtainable by a process as characterized here, is the subject of the present invention. By making available this directly shaped tablet disadvantages as described above can be overcome in a simple manner.

### DETAILED DESCRIPTION OF THE INVENTION

While the making and using of various embodiments of the present invention are discussed in detail below, it should be appreciated that the present invention provides more applicable inventive concepts than described here in detail. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “a homogenous melt, or mixture or form” refers to the various compositions that can be made by extruding the made-up source material.

As used herein, the term “heterogeneously homogeneous composite” refers to a material composition having at least two different materials that are evenly and uniformly distributed throughout the volume and which are prepared of the one or more APIs and the one or more pharmaceutically acceptable excipients, including a pretreated PVA into a composite material.

As used herein, “bioavailability” is a term meaning the degree to which a drug becomes available to the target tissue after being administered to the body.

Poor bioavailability is a significant problem encountered in the development of pharmaceutical compositions, particularly those containing an active ingredient that is not highly soluble.

As used herein, the phrase “pharmaceutically acceptable” refers to molecular entities, compositions, materials, excipients, carriers, and the like that do not produce an allergic or similar untoward reaction when administered to humans in general.

As used herein, “pharmaceutically acceptable carrier” or “pharmaceutically acceptable materials” includes any and all solvents, dispersion media, coatings, antibacterial and antifungal agents, isotonic and absorption delaying agents, and the like. The use of such media and agents for pharmaceutical active substances is well known in the art.

The API (active pharmaceutical ingredient) may be found in the form of one or more pharmaceutically acceptable salts, esters, derivatives, analogs, prodrugs, and solvates thereof. As used herein, a “pharmaceutically acceptable salt” is understood to mean a compound formed by the interaction of an acid and a base, the hydrogen atoms of the acid being replaced by the positive ion of the base.

As used herein, “poorly soluble” refers to having a solubility means the substance needs ≥100 ml solvent to dissolve 1 g substance.

A variety of administration routes are available for delivering the APIs to a patient in need. The particular route selected will depend upon the particular drug selected, the weight and age of the patient, and the dosage required for therapeutic effect. The pharmaceutical compositions may conveniently be presented in unit dosage form. The APIs suitable for use in accordance with the present disclosure, and their pharmaceutically acceptable salts, derivatives, analogs, prodrugs, and solvates thereof, can be administered alone, but will generally be administered in admixture with a suitable pharmaceutical excipient, diluent, or carrier selected with regard to the intended route of administration and standard pharmaceutical practice.

The excipients and adjuvants that may be used in the presently disclosed compositions and composites, while potentially having some activity on their own, for example, antioxidants, are generally defined for this application as compounds that enhance the efficiency and/or efficacy of the effective ingredients. It is also possible to have more than one effective ingredient in a given solution, so that the particles formed contain more than one effective ingredient.

As stated, excipients and adjuvants may be used to enhance the efficacy and efficiency of the APIs dissolution.

Depending on the desired administration form the formulations can be designed to be suitable in different release models, which are well known to the skilled person, as there are immediate, rapid or extended release, delayed release or for controlled release, slow release dosage form or mixed release, including two or more release profiles for one or more active pharmaceutical ingredients, timed release dosage form, targeted release dosage form, pulsatile release dosage form, or other release forms.

The resulting composites or compositions disclosed herein may also be formulated to exhibit enhanced dissolution rate of a formulated poorly water-soluble drug.

The United States Pharmacopeia-National Formulary mandates that an acceptable polyvinyl alcohol for use in pharmaceutical dosage forms must have a percentage of hydrolysis between 85 and 89%, as well as a degree of polymerization between 500 and 5000. The degree of polymerization (DM) is calculated by the equation:

DM=(Molar Mass)/((86)−(0,42(the degree of hydrolysis)))

The European Pharmacopoeia mandates that an acceptable polyvinyl alcohol for use in pharmaceutical dosage forms must have an ester value not greater than 280 and a mean relative molecular mass between 20,000 and 150,000. The percentage of hydrolysis (H) can be calculated from the following equation:

H=((100−(0,1535)(EV))/(100−(0,0749)(EV)))×100

Where EV is the ester value of the polymer. Thus, only polymers with a percentage of hydrolysis greater than 72.2% are acceptable according to the European Pharmacopoeia monograph.

As already mentioned above, commercial polyvinyl alcohols in particulate form have poor flow behavior, especially if they are characterized by low viscosities (measured in a 4% aqueous solution at 20° C.). Accordingly, these powders have no continuous trouble-free flow. However, the latter is a prerequisite for a uniform feed to the processing of such powder materials.

Theoretically, powders, whose particle shapes are rather round and spherical, in general have the best flow behavior. Accordingly, in the past, attempts have been made to produce polyvinyl alcohol powders already directly by its synthesis with spherical particles. For example, from DE 38 11 201A a method is known for producing of spherical particles by suspension polymerization. However, this reaction requires a special adjustment of the reaction conditions. In addition, this reaction has to be followed by a hydrolysis reaction. With different particle sizes, it is difficult to achieve a uniform degree of hydrolysis of the polymer particles. By this method, polyvinyl alcohol powders are produced having viscosities of 80 mPa·s or higher.

Therefore, for the production of polyvinyl alcohol powders, which are comparable with those of the present invention, this method provides no alternative, especially as here PVA grades are desirable having viscosities of ≥40 mPa·s.

Now, it was found, that these polyvinyl alcohol grades having viscosities of ≥40 mPa·s are also suitable to be manufactured by melt extrusion, if they are pretreated as disclosed in the following and a homogenously dispersed solid solution of pharmaceutical active ingredient in polyvinyl alcohol can be produced by extrusion and the applied PVA powder can be fed without problems into the feeder.

In this way also poorly soluble pharmaceutical active ingredients (from BCS class II and IV) can be homogeneously mixed with PVA to build a solid dispersion. Furthermore, it was found by experiments that PVA in the different degrees of hydrolysis having viscosities of ≥40 mPa·s can be homogeneously mixed by melt extrusion with poorly soluble active ingredients, especially with PVA that is in accordance with the European Pharmacopoeia monograph and which is a pharmaceutically acceptable PVA with hydrolysis grades greater than 72.2%, and especially which includes grades of PVA that are pharmaceutically acceptable by either the USP (hydrolysis between 85-89%) or Ph. Eur. (hydrolysis grades greater than 72.2%). These PVA qualities have a molecular weight in the range of 14,000 g/mol to 250,000 g/mol.

Directly shaped tablet compositions according to the invention may comprise at least a pharmaceutical active ingredient combined with a PVA that is pharmaceutically acceptable, which is combined with another pharmaceutically acceptable polymer. Such pharmaceutically acceptable polymer can also be selected from the group of hydrophilic polymers and can be a primary or secondary polymeric carrier that can be included in the composition disclosed herein and including polyethylene-polypropylene glycol (e.g. POLOXAMER™), carbomer, polycarbophil, or chitosan, provided that they are as free-flowing powder and are extrudable polymers.

Hydrophilic polymers for use with the present invention may also include one or more of hydroxypropyl methylcellulose, carboxymethylcellulose, hydroxypropyl cellulose, hydroxyethyl cellulose, methylcellulose, natural gums such as gum guar, gum acacia, gum tragacanth, or gum xanthan, and povidone. Hydrophilic polymers also include polyethylene oxide, sodium carboxymethycellulose, hydroxyethyl methyl cellulose, hydroxymethyl cellulose, carboxypolymethylene, polyethylene glycol, alginic acid, gelatin, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

In general, it must be considered that there are special requirements for polymers used as hot melt extrusion excipients:

The polymer must be thermoplastic, must have a suitable glass transition temperature and a high thermal stability. The polymer must have no toxic properties and must have a high biocompatibility, etc. Therefore, pharmaceutical grades of polyvinyl alcohol (PVA), which are chosen here for the preparation of formulations comprising active ingredients by hot melt extrusion, are those having a low viscosity.

Polyvinyl alcohol (PVA) is a synthetic polymer, which is produced by polymerization of vinyl acetate and partial hydrolysis of the resulting esterified polymer. As already mentioned above, chemical and physical properties of polyvinyl alcohol, such as viscosity, solubility, thermal properties, etc. are very depending on its degree of polymerization, chain length of PVA polymer, and the degree of hydrolysis.

PVA can be used for the production of different formulations for various modes of administration to treat a variety of disorders. Accordingly, PVA is processed in a wide range of pharmaceutical dosage forms, including ophthalmic, transdermal, topical, and especially, oral application forms.

As mentioned above, it is for the successful industrial processing of a solid dosage form in 1) a physical mixing process 2) an extrusion process 3) a directly shaping process into tablet, also necessary that a uniform continuous metering is possible in the extruder, direct shaping equipment for tablet.

As already mentioned above, for the successful industrial processing to a solid dosage form are not only
1) a physical mixing process,
2) an extrusion process, and
3) a direct shaping process to form tablets using a suitable shaping equipment

required, but it is also necessary that a uniform continuous dosing in the extruder is possible.

Now it was found by experiments, that for hot melt extrusion and directly shaping of extrudate into tablet, polyvinyl alcohol (PVA) having pharmaceutical grade, selected from the group: PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88, are for this method suitable.

It is well known that the gel layer on the surface of PVA compressed tablet blocks the release of a contained poorly water-soluble API, and may promote recrystallization of the API within the tablets, because the API suffers a super saturated state inside of the compressed tablet. But it is found now, that the directly shaped tablet based on PVA can overcome this disadvantage.

Surprisingly, it was found by experiments special tablet compositions by which this problem can be solved.

These compositions are
1. based on a PVA/API extrudate, and contain no binder material,
2. may contain plasticizer, antioxidants, stabilizing agents, solubility enhancing agents, and pH control agents within the extrudate.

The use of the extruded compositions of the invention allows the disintegration of the tablets formed directly therefrom and protects the API against recrystallization. However, this is not the only beneficial effect of the extruded compositions of the present invention. Surprisingly, they result in the directly shaped tablets having an anti-alcohol-induced dose dumping effect.

### EXAMPLES

Even without any further explanations, it is assumed that a person skilled in the art can make use of the above description in its widest scope. The preferred embodiments and examples are therefore to be regarded merely as descriptive but in no way limiting disclosures.

For better understanding and for illustration, examples are given below which are within the scope of protection of the present invention. These examples also serve for the illustration of possible variants.

The complete disclosure of all applications, patents and publications mentioned above and below are incorporated by reference in the present application and shall serve in cases of doubt for clarification.

It goes without saying that, both in the examples given and also in the remainder of the description, the quoted percentage data of the components present in the compositions always add up to a total of 100% and not more. Given temperatures are measured in ° C.

Before extrusion, PVA is physically blended with active ingredients in an amount of 20-60% by weight, with or without additional excipients. The mixture is extruded under suitable conditions (depends on API) and directly extruded into tablet, which is characterized regarding to the feasibility of directly shaping into tablets, homogeneity of API within the tablet and dissolution performance of tablets with different concentrations of alcohol according to the FDA standard method for “anti-alcohol induced dose dumping”.

Methods and Materials

1. Raw Materials and Manufacturing Method

1.1 Materials

Raw Material:
Poly vinyl alcohol 4-88, excipient EMPROVE® exp Ph Eur, USP, JPE, Article No. 1.41350, Merck KGaA, Darmstadt, Germany
Itraconazole, active ingredient, Selectchemie, AG, Germany

1.2 Experiments and Characterization Methods

1.2.1 Extrusion Process

Equipment:
Physical blend of composition for hot melt extrusion, including active ingredients: TURBULA® Shaker-Mixer
Brabender® Mini-Compounder KETSE 12/36 D
Brabender® Pelletizer
The mixture of PVA and active ingredient were blended using TURBULA® Shaker-Mixer homogeneously (the concentration of polymer and active ingredient depends on the types and physical properties of them). The mixture was then loaded into the extruder with well designed extrusion parameters, such as feeding rate, screw design, screw speed, extrusion temperature etc. The set up of those parameters depend also on the types and physical properties of polymer and active ingredients. The extrudate was directly shaped into tablet form.

1.2.2 Directly Shaping Process of Tablet

At first, the extruded material was cut into small pieces with a length of about 2.5 cm directly after leaving the hot nozzle (diameter: 5 mm). Cutting was done with a nipper and forceps, both made of stainless steel. While still in hot condition, the extruded material was then filled into an 11 mm round tablet mold die set and compressed by hand using a 5 kg weight. After compressing, the tablet was pushed out of the mold by using the punch and the weight of the tablet was checked.

1.2.3 Dissolution

For the real time dissolution performance, we use the following equipments:

System 1:
Sotax AT 7 on/offline
Pumpe CY-7-50
Fraktionssammler: C613 14 Kanal 3 Wege Ventilbalken für Reagenzgläser
Agilent 8453 Photometer

System 2
Sotax AT 7 on/offline
Pumpe CP 7-35
Fraktionssammler: C 613 14 Kanal 3 Wege Ventilbalken für Vials
Photometer Analytik Jena Specord 200 plus

2. Results

2.1 Homogeneity of API within the Tablet

It was planned to extrude 30% itraconazole and 70% PVA together and directly shaped them into tablets. Following table shows the homogeneity of itraconazole within the directly shaped tablet.

TABLE 1
Homogeneity of itraconazole within the directly shaped tablet

| | | | Percentage of | |
|---|---|---|---|---|
| | Recovery of | Itraconazole | detected | |
| | itraconazole [%] | (HPLC) [mg] | itraconazole (%) | STDEV |
| Tablet 1 | 99.54 | 213.05 | 29.9 | 0.515 |
| Tablet 2 | 100.72 | 215.58 | 30.2 | |
| Tablet 3 | 96.37 | 211.56 | 28.9 | |
| Tablet 4 | 100.27 | 220.12 | 30.1 | |
| Tablet 5 | 101.10 | 195.07 | 30.3 | |
| Tablet 6 | 100.19 | 193.29 | 30.1 | |

This table shows a homogenous distribution of API: after the extrusion and directly shaping of tablets, no degradation of API is observed and the API has a homogenous distribution within each tablet.

1.2 Anti-Alcohol Induced Dose Dumping Dissolution

The dissolution performance both of the milled powder and of directly shaped tablets is evaluated under the same conditions and the same dissolution medium following the FDA standard method is applied: 0.1 M HCL without ethanol, 0.1 M HCL with 10% ethanol, 0.1 M HCL with 20% ethanol, 0.1 M HCL with 40% ethanol.

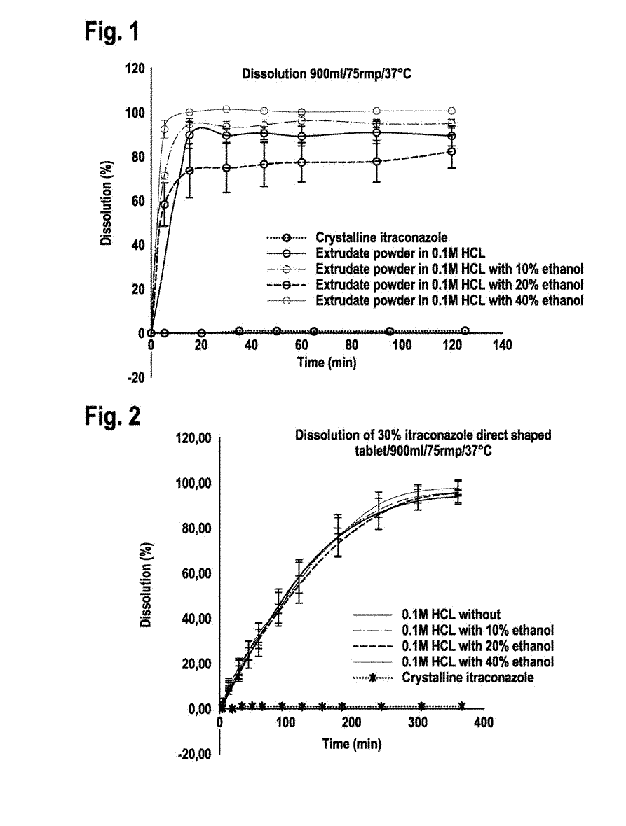

FIG. 1: alcohol-induced-dose dumping from the milled powder (not the tablet) as negative example

For the powder form significant different dissolution behaviour is observed with different ethanol concentrations. This means, that an alcohol-induced-dose dumping effect happens with the milled powder (negative).

FIG. 2: shows anti-alcohol induced-dose dumping effect with our directly shaped tablet from PVA extrudate: no significant difference was observed using solutions containing different alcohol concentrations.

For directly shaped tablet (which has the same composition as the milled powder but just is in a different form): the same dissolution process is repeated with this tablet based on PVA. Surprisingly, it is found that there is no significant difference between the dissolution with different ethanol concentrations. This means, that there is an anti-alcohol induced-dose dumping effect if the composition is in the form of a shaped tablet produced from the PVA extrudate. If the PVA extrudate is used in powder form, it loses the anti-alcohol induced-dose dumping effect.

2.3 Long Term Stability of Tablet

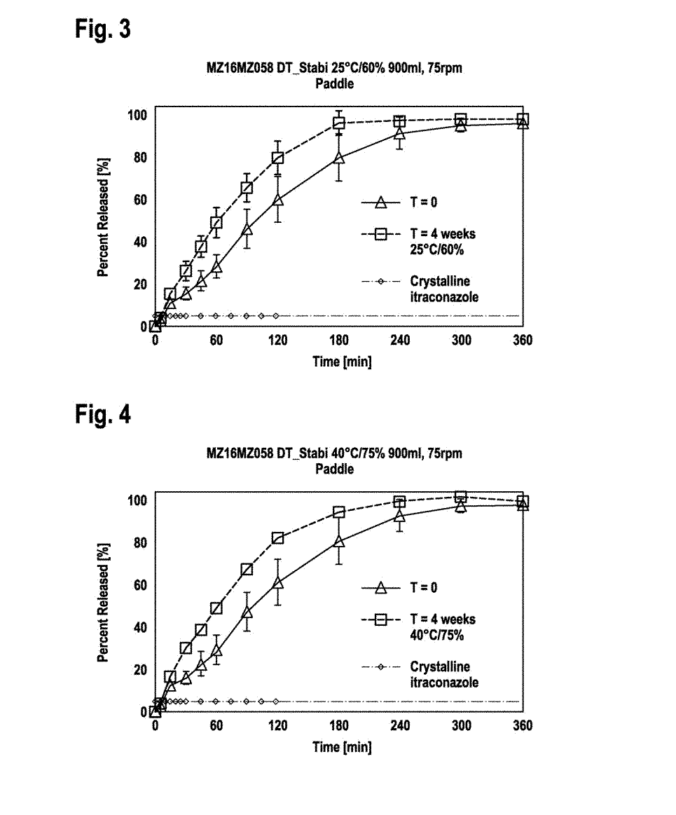

FIG. 3: shows the dissolution of tablets, which were stored under the condition 25° C./60% for more than 6 months.

FIG. 4: shows the dissolution of tablets, which were stored under the condition 40° C./75% more than 6 months.

Stability tests for one month with these directly shaped tablets under different conditions show no re-crystallization of the contained API. Repeated dissolution experiments show the same results.

2.4 Summary

Advantages of investigated powders and compositions:
1. The directly shaped tablets based on PVA extrudate according to the invention has anti-alcohol-induced dose dumping effect, even for the poorly water-soluble API from BCS class II and IV.
2. Directly shaped tablets of the invention can be produced under reduced costs using hot melt extrusion for the material and the formulation processing.
3. The produced tablets are long term storage stable and transport-stable.

1. Directly shaped tablet produced from a polyvinyl alcohol (PVA) based extrudate with anti-alcohol-induced dose dumping effect.

2. Tablet according to claim 1, which is hot melt extruded or melt extruded.

3. Tablet according claim 1, produced from a polyvinyl alcohol grade having a viscosity ≤40 mPa·s, the viscosity being measured on 4% w/v aqueous solution at 20° C. DIN 53015.

4. Tablet according to claim 1, produced from a polyvinyl alcohol grade which is selected from the group PVA 3-88, PVA 4-88, PVA 5-74, PVA 5-88, PVA 8-88, and PVA 18-88.

5. A tablet composition with anti-alcohol-induced dose dumping effect, comprising extruded polyvinyl alcohol, which is extruded and homogeneously milled together with at least one active pharmaceutical ingredient (API), and optionally one or more additives, selected from the group plasticizer, antioxidants, stabilizing agents, solubility-enhancing agents, and pH control agents, whereby the directly shaped tablet is storage and transport-stable, showing an anti-alcohol-induced dose dumping effect.

6. A tablet composition according to claim 5, comprising a polyvinyl alcohol grade, which is selected from the group PVA 3-88, PVA 4-88, PVA 5-74, PVA 5-88, PVA 8-88, and PVA 18-88, and having a viscosity ≤40 mPa·s, the viscosity being measured on 4% w/v aqueous solution at 20° C. DIN 53015.

7. A tablet composition according to claim 5, which is hot melt extruded or melt extruded.

8. A process for producing a solid pharmaceutical directly shaped tablet from extrudate, characterized in that the mixture of ingredients comprises the powdered polyvinyl alcohol claim 1.
